(19) United States
(12) Patent Application Publication
(10) Pub. No.: US 2017/0136101 A1
(43) Pub. Date: May 18, 2017

(54) Methods for preventing and/or treating nasal polyps and rhinosinusitis

(71) Applicant: Noveome Biotherapeutics, Inc., Pittsburgh, PA (US)

(72) Inventor: Larry R Brown, Newton, MA (US)

(73) Assignee: Noveome Biotherapeutics, Inc., Pittsburgh, PA (US)

(21) Appl. No.: 15/362,951

(22) Filed: Nov. 29, 2016

Related U.S. Application Data
(62) Division of application No. 14/183,599, filed on Feb. 19, 2014, now abandoned.
(60) Provisional application No. 61/768,430, filed on Feb. 23, 2013.

(51) Int. Cl. A61K 38/46; A61K 38/57; A61K 9/12; A61K 38/18; A61K 9/00; A61K 9/08

(52) U.S. Cl. CPC A61K 38/465; A61K 9/0043; A61K 9/08; A61K 9/12; A61K 38/1866; A61K 38/1858; A61K 38/1841; A61K 38/57

(57) ABSTRACT

The invention is directed to methods for preventing and/or treating nasal polyps. The invention is further directed to methods for preventing and/or treating rhinosinusitus. The invention is further directed to reducing inflammation of the paranasal sinuses. The invention is further directed to methods for preventing and/or treating nasal polyps and/or rhinosinusitis by administering to a subject suffering from such conditions, or at risk of developing such conditions, novel cellular factor-containing solution compositions (referred to herein as “CFS” compositions), including novel immediate-release, targeted-release, and sustained-release (SR) cellular factor-containing solution compositions (referred to herein as “SR-CFS” compositions)

### FIELD OF THE INVENTION

The field of the invention is directed to methods for preventing and/or treating nasal polyps. The field of the invention is further directed to methods for preventing and/or treating rhinosinusitus. The field of the invention is further directed to reducing inflammation of the paranasal sinuses. The field of the invention is further directed to methods for preventing and/or treating nasal polyps and/or rhinosinusitis and/or inflammation of the paranasal sinuses by administering to a subject suffering from such conditions, or at risk of developing such conditions, novel cellular factor-containing solution compositions (referred to herein as “CFS” compositions), including novel immediate-release, targeted-release, and sustained-release (SR) cellular factor-containing solution compositions (referred to herein as “SR-CFS” compositions).

### BACKGROUND OF THE INVENTION

Rhinosinusitus is a condition involving inflammation in one or more of the paranasal sinuses. Acute sinusitis is caused by an infection and generally resolves with antibiotics. Chronic sinusitis is sinonasal inflammation lasting for more than 12 weeks and may be caused by multiple factors. Recurrent sinusitis and chronic sinusitis can cause long-term symptoms that can negatively impact a person's quality of life. Chronic inflammation of the mucosal lining in the nose and sinuses may lead to the formation of nasal polyps in severe cases. Nasal polyposis is a chronic inflammatory disease of the upper airway characterized histologically by the infiltration of inflammatory cells such as eosinophils and neutrophils. Nasal polyps and rhinosinusitis affect millions of people in both the United States and around the world. Sinusitis is one of the most common diseases diagnosed in the United States. Patients who have chronic rhinosinusitis frequently develop sinonasal polyps. Nasal polyps are the most common non-cancerous tumors of the nasal cavity. Approximately 30% of patients with nasal polyps test positive for environmental allergies. The prevalence of nasal polyps is increased in children with cystic fibrosis and persons with known aspirin hypersensitivity. Several hypotheses as to the etiology of nasal polyps have been suggested regarding the underlying mechanisms including chronic infection, aspirin intolerance, alteration in aerodynamics of the nasal cavity resulting in trapping of pollutants, epithelial disruptions, epithelial cell defects/gene deletions (CFTR gene), and inhalant or food allergies.

Nasal polyps are typically treated by surgical removal. However, surgical interventions may lead to unsatisfactory healing and may cause complications due to scar formation, because mucosal wound healing also may be impaired as a result of poorly defined factors in the inflamed mucosa. Furthermore, a study of 118 patients showed that 60% developed recurrent polyposis after surgery. (Wynn R, Har-El G. Recurrence rates after endoscopic sinus surgery for massive sinus polyposis. Laryngoscope. 2004 May; 114(5):811-3, Eitan, Y, Shvero J, Drusd T, Tamir R, Hadar T, Recurrence of nasal polyps after functional endoscopic sinus surgery, Conexiuni medicale 2009, 4 (16) 27-29, and Akhtar S, Ikram M, Azam I, Dahri T, Factors associated with recurrent nasal polyps: A tertiary care experience, JPMA 60:102; 2010.). Eosinophils comprise more than 60% of the cell population. Besides eosinophils, mast cells and activated T cells are also increased. An increased production of cytokines and chemokines such as GM-CSF, IL-5, RANTES and eotaxin contribute to eosinophil migration and survival. Increased levels of IL-8 can induce neutrophil infiltration. Increased expression of VEGF and its upregulation by TGFβ can contribute to edema and increased angiogenesis in nasal polyps. TGFβ can also modulate fibroblast function and thus contribute to eosinophil infiltration and stromal fibrosis. Other mediators like albumin, histamine and the immunoglobulins IgE and IgG are also increased in nasal polyps. In addition, the local production of IgE in nasal polyps can contribute to the increased recurrence of nasal polyps via the IgE-mast cell-Fc ε RI cascade. Finally, mast cell/T cell/epithelial cell/fibroblast interactions can contribute to the persistent eosinophilic inflammation seen in nasal polyps. Researchers have shown that there is an upregulation of mesenchymal-epithelial transition factor, periostin, and protein phosphatase 1 regulatory subunit 9B. TGFβ1 has also been implicated in the occurrence of non-allergic nasal polyps. (Platt et al, Current Opinion in Allergy and Clinical Immunology, 2009, 9:23-28).

Systemic steroids, which are less well studied, have an effect on all types of symptoms and pathology, including the sense of smell. This type of treatment, which can serve a “medical polypectomy,” is only used for short-term improvement due to the risk of adverse effects. Individualized management of nasal polyposis may use long-term topical steroids, short-term systemic steroids, as well as surgery, in various combinations. Exactly how these therapies, which differ in their control of various symptoms, are optimally combined is not yet well established.

Topically applied steroids are the therapeutic modality that has been best studied in controlled trials. It reduces rhinitis symptoms, improves nasal breathing, reduces the size of polyps and the recurrence rate, but it has a negligible effect on improving the sense of smell and on any sinus pathology. Topical steroids can, as long-term therapy, be used alone in mild cases, or combined with systemic steroids/surgery in severe cases.

A treatment that reduces or eliminates the inflammatory stimuli that result in nasal polyps would have a major impact on their treatment. Further, a treatment that could eliminate the need for surgical intervention is also desirable. Accordingly, it is an object of the instant invention to provide such a treatment option.

### BRIEF SUMMARY OF THE INVENTION

Applicants have discovered that Amnion-derived Cellular Cytokine Solution (ACCS) (for details see U.S. Pat. Nos. 8,058,066 and 8,088,732, both of which are incorporated herein by reference) exhibits many anti-inflammatory properties. Therefore, ACCS, delivered as a liquid nasal spray or incorporated into nasal packing inserted after surgical removal of polyps, would be expected to be an effective means of preventing the development of or reoccurrence of nasal polyps by placing the composition proximal to the sites of inflammation in the nasal passages. The administration of ACCS to the nasal mucosa will clearly be safer than chronic administration of steriods, and offers the real possibility that it will be able to effectively inhibit the inflammatory response on the cellular level. Unlike steroids, ACCS would not be expected to affect the sense of smell.

It is important to note that surgical removal of nasal polyps is not a permanent cure to the disease. Nasal polyps may often recur within one year after surgery (see, for example, Wynn R, Har-El G. Recurrence rates after endoscopic sinus surgery for massive sinus polyposis. Laryngoscope. 2004 May; 114(5):811-3 , Eitan, Y, Shvero J, Drusd T, Tamir R, Hadar T, Recurrence of nasal polyps after functional endoscopic sinus surgery, Conexiuni medicale 2009, 4 (16) 27-29, and Akhtar S, Ikram M, Azam I, Dahri T, Factors associated with recurrent nasal polyps: A tertiary care experience, JPMA 60:102; 2010). Nasal administration of ACCS offers the possibility that the inflammatory pathways can be prevented which will obviate or delay the need for costly and painful surgical removal of the polyps and obviate or delay the need for expensive additional surgical procedures to remove recurring nasal polyps.

To prevent or treat these conditions, the instant invention provides novel cellular factor-containing solution (CFS) compositions, including ACCS, for use in the described methods. The instant invention also provides novel sustained-release cellular factor-containing solution (SR-CFS) compositions, including SR-ACCS, for use in the methods. The instant invention also provides for nasal spray administration of the CFC compositions, as well as delivery using sustained-release nasal packing inserted after surgical removal. Because the cellular factors are present in the compositions at levels comparable to physiological levels found in the body, they are optimal for use in therapeutic applications which require intervention to support, initiate, replace, accelerate or otherwise influence biochemical and biological processes involved in the treatment and/or healing of disease and/or injury. In the case of the SR-CFS compositions, the cellular factors are released slowly over time to provide a continual, consistent physiologic level of such factors to reduce local inflammation and therefore optimize healing and/or recovery. Detailed information about the compositions used in the methods can be found in U.S. Pat. Nos. 8,058,066 and 8,088,732, both of which are incorporated herein by reference.

Accordingly, a first aspect of the invention is method for preventing nasal polyps in a patient in need thereof comprising administering to the patient a therapeutically effective amount of a CFS composition.

A second aspect of the invention is a method for preventing rhinosinusitis in a patient in need thereof comprising administering to the patient a therapeutically effective amount of a CFS composition.

A third aspect of the invention is a method for treating nasal polyps in a patient in need thereof comprising administering to the patient a therapeutically effective amount of a CFS composition.

A fourth aspect of the invention is a method for treating rhinosinusitis in a patient in need thereof comprising administering to the patient a therapeutically effective amount of a CFS composition.

A fifth aspect of the invention is a method for reducing inflammation of the paranasal sinuses associated with the development of nasal polyps or rhinosinusitis in a patient in need thereof comprising administering to the patient a therapeutically effective amount of a CFS composition such that inflammation of the paranasal sinuses is reduced.

A specific embodiment of the fifth aspect of the invention is wherein in the inflammation of the paranasal sinuses associated with the development of nasal polyps or rhinosinusitis is the result of one or more of a condition or circumstance selected from the group consisting of chronic infection, aspirin intolerance, alteration in aerodynamics of the nasal cavity resulting in trapping of pollutants, epithelial disruptions, epithelial cell defects/gene deletions (CFTR gene), inhalants and food allergies.

Further specific embodiments of aspects one through five of the invention are ones wherein the CFS composition is ACCS. In a particular embodiment, the ACCS is formulated for intranasal administration. In another particular embodiment, the intranasal administration is aerosol or spray administration. In still another particular embodiment, the ACCS is contained in a surgical packing material.

The above-described aspects and embodiments of the invention are not intended to be limiting, but rather exemplary. Skilled artisans will recognize that additional aspects and embodiments of the invention, though not explicitly or specifically described, are contemplated and encompassed by the teachings and examples set forth in the specification.

### Definitions

As defined herein “isolated” refers to material removed from its original environment and is thus altered “by the hand of man” from its natural state.

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cytokine secreting cells” or “ECS cells” means a population of cells derived from the extraembryonic tissue which have the characteristics of secreting a unique combination of physiologically relevant cytokines in a physiologically relevant temporal manner into the extracellular space or into surrounding culture media and which have not been cultured in the presence of any animal-derived products, making them and cell products derived from them suitable for human clinical use. In a preferred embodiment, the ECS cells secrete the cytokines VEGF, Angiogenin, PDGF and TGF432 and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2. The ECS cells may optionally express Thymosin β4.

As used herein, the term “amnion-derived multipotent progenitor cell” or “AMP cell” means a specific population of ECS cells that are epithelial cells derived from the amnion. In addition to the characteristics described above for ECS cells, AMP cells have the following characteristics. They have not been cultured in the presence of any non-human animal-derived products, making them and cell products derived from them suitable for human clinical use. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “serum-free” when referring to certain compositions, growth conditions, culture media, etc. described herein, is meant that no animal-derived serum (i.e. no non-human) is used in the preparation, growth, culturing, expansion, storage or formulation of the certain composition or process.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from ECS cells, including AMP cells.

As used herein, the term “cellular factor-containing solution” or “CFS” composition means a composition having physiologic concentrations of one or more protein factors. CFS compositions include conditioned media derived from ECS cells, amnion-derived cellular cytokine solution compositions (see definition below), physiologic cytokine solution compositions (see definition below), and sustained release formulations of such CFS compositions.

As used herein, the term “amnion-derived cellular cytokine solution” or “ACCS” means conditioned medium that has been derived from AMP cells or expanded AMP cells.

As used herein, the term “physiologic cytokine solution” or “PCS” composition means a composition which is not cell-derived and which has physiologic concentrations of VEGF, Angiogenin, PDGF and TGFβ2, TIMP-1 and TIMP-2.

As used herein, the term “suspension” means a liquid containing dispersed components, i.e. cytokines. The dispersed components may be fully solubilized, partially solubilized, suspended or otherwise dispersed in the liquid. Suitable liquids include, but are not limited to, water, osmotic solutions such as salt and/or sugar solutions, cell culture media, and other aqueous or non-aqueous solutions.

The term “lysate” as used herein refers to the composition obtained when cells, for example, AMP cells, are lysed and optionally the cellular debris (e.g., cellular membranes) is removed. This may be achieved by mechanical means, by freezing and thawing, by sonication, by use of detergents, such as EDTA, or by enzymatic digestion using, for example, hyaluronidase, dispase, proteases, and nucleases.

The term “physiologic” or “physiological level” as used herein means the level that a substance in a living system is found and that is relevant to the proper functioning of a biochemical and/or biological process.

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

The term “cell product” or “cell products” as used herein refers to any and all substances made by and secreted from a cell, including but not limited to, protein factors (i.e. growth factors, differentiation factors, engraftment factors, cytokines, morphogens, proteases (i.e. to promote endogenous cell delamination, protease inhibitors), extracellular matrix components (i.e. fibronectin, etc.).

The term “therapeutically effective amount” means that amount of a therapeutic agent necessary to achieve a desired physiological effect (i.e. accelerate wound healing).

As used herein, the term “pharmaceutically acceptable” means that the components, in addition to the therapeutic agent, comprising the formulation, are suitable for administration to the patient being treated in accordance with the present invention.

As used herein, the term “therapeutic component” means a component of the composition which exerts a therapeutic benefit when the composition is administered to a subject.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

As used herein, the term “tissue” refers to an aggregation of similarly specialized cells united in the performance of a particular function.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time-release capsules and the like.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral and intrasternal injection or infusion.

As used herein, the term “aerosol” means a cloud of solid or liquid particles in a gas.

The terms “particles”, “aerosolized particles”, and “aerosolized particles of formulation” are used interchangeably herein and shall mean particles of formulation comprised of any pharmaceutically active ingredient, preferably in combination with a carrier, (e.g., a pharmaceutically active respiratory drug and carrier). The particles have a size which is sufficiently small such that when the particles are formed they remain suspended in the air or gas for a sufficient amount of time such that a patient can inhale the particles into the patient's lungs.

As used herein, the term “nebulizer” means a device used to reduce a liquid medication to extremely fine cloudlike particles (i.e. an aerosol). A nebulizer is useful in delivering medication to deeper parts of the respiratory tract. Nebulizers may also be referred to as atomizers and vaporizers.

The term “intranasal” or “intranasal delivery” or “intranasal administration” as used herein means delivery within or administered by way of the nasal structures.

The term “immediate-release” as used herein means that all of the pharmaceutical agent(s) is released into solution and into the biological orifice or blood or cavity etc. at the same time.

The term “targeted-release” as used herein means that the pharmaceutical agent is targeted to a specific tissue, biological orifice, tumor site or cavity, etc.

The terms “sustained-release”, “extended-release”, “time-release”, “controlled-release”, or “continuous-release” as used herein means an agent, typically a therapeutic agent or drug, that is formulated to dissolve slowly and be released over time.

As used herein, the term “nasal polyp” means sac-like growths of inflamed tissue lining the nose (nasal mucosa) or paranasal sinuses.

As used herein, the term “rhinosinusitis” or “sinusitis” means a condition involving inflammation in one or more of the paranasal sinuses. Acute sinusitis is caused by an infection and usually resolves with medical therapy including antibiotics. Chronic sinusitis is sinonasal inflammation lasting for more than 12 weeks and can be cause by multiple factors. Recurrent sinusitis and chronic sinusitis can cause long-term symptoms that can negatively impact a person's quality of life. Chronic inflammation of the mucosal lining in the nose and sinuses may lead to the formation of nasal polyps in severe cases.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e. contusion, penetrating), thermal, chemical, electrical, radiation, concussive and incisional injuries; elective injuries such as operative surgery and resultant incisional hernias, fistulas, etc.; acute wounds, chronic wounds, infected wounds, and sterile wounds, as well as wounds associated with disease states (i.e. ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

As used herein the term “standard animal model” refers to any art-accepted animal model in which the compositions of the invention exhibit efficacy.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Compositions and Methods of Making Compositions

Detailed information and methods on the preparation of AMP cell compositions, generation of ACCS, generation of pooled ACCS, detection of cytokines in non-pooled and pooled ACCS using ELISA, generation of PCS compositions, and generation of sustained-release CFS compositions can be found in U.S. Pat. Nos. 8,058,066 and 8,088,732, both of which are incorporated herein by reference.

The invention provides for an article of manufacture comprising packaging material and a pharmaceutical composition of the invention contained within the packaging material, wherein the pharmaceutical composition comprises CFS compositions, including ACCS. The packaging material comprises a label or package insert which indicates that the CFS compositions, including ACCS, contained therein can be used for therapeutic applications such as, for example, preventing or treating nasal polyps.

### Formulation, Dosage and Administration of CFS Compositions

Compositions comprising CFS compositions may be administered to a subject to provide various cellular or tissue functions, for example, to prevent or treat nasal polyps. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may also be administered to the recipient in one or more physiologically acceptable carriers. Carriers for CFS compositions may include but are not limited to solutions of normal saline, phosphate buffered saline (PBS), lactated Ringer's solution containing a mixture of salts in physiologic concentrations, or cell culture medium.

In addition, one of skill in the art may readily determine the appropriate dose of the CFS compositions for a particular purpose. A preferred dose is in the range of about 0.1-to-1000 micrograms per square centimeter of applied area. Other preferred dose ranges are 1.0-to-50.0 micrograms/applied area. In a particularly preferred embodiment, it has been found that relatively small amounts of the CFS compositions are therapeutically useful. One exemplification of such therapeutic utility is the ability for ACCS (including pooled ACCS) to accelerate wound healing (for details see U.S. Publication No. 2006/0222634 and U.S. Pat. No. 8,187,881, both of which are incorporated herein by reference). One of skill in the art will also recognize that the number of doses to be administered needs also to be empirically determined based on, for example, severity and type of disease, disorder or injury being treated; patient age, weight, sex, health; other medications and treatments being administered to the patient; and the like. For example, in a preferred embodiment, one dose is sufficient to have a therapeutic effect (i.e. prevent or treat nasal polyps). Other preferred embodiments contemplate, 2, 3, 4, or more doses for therapeutic effect.

One of skill in the art will also recognize that number of doses (dosing regimen) to be administered needs also to be empirically determined based on, for example, severity and type of injury, disorder or condition being treated; patient age, weight, sex, health; other medications and treatments being administered to the patient; and the like. In addition, one of skill in the art recognizes that the frequency of dosing needs to be empirically determined based on similar criteria. In certain embodiments, one dose is administered every day for a given number of days (i.e. once a day for 7 days, etc.). In other embodiments, multiple doses may be administered in one day (every 4 hours, etc.). Multiple doses per day for multiple days is also contemplated by the invention.

In further embodiments of the present invention, at least one additional agent may be combined with the CFS compositions. Such agents may act synergistically with the CFS compositions of the invention to enhance the therapeutic effect. Such agents include but are not limited to growth factors, cytokines, chemokines, antibodies, inhibitors, antibiotics, immunosuppressive agents, steroids, anti-fungals, anti-virals or other cell types (i.e. stem cells or stem-like cells, for example AMP cells). Inactive agents include carriers, diluents, stabilizers, gelling agents, delivery vehicles, ECMs (natural and synthetic), scaffolds, and the like. When the CFS compositions are administered conjointly with other pharmaceutically active agents, even less of the CFS compositions may be needed to be therapeutically effective.

### Aerosol Compositions

Methods for creating aerosol compositions are well known to skilled artisans. Specifics can be found in “Drug Delivery to the Lung” By Hans Bisgaard, Christopher O'Callaghan, Gerald C. Smaldone, published by Informa Health Care, 2001, and elsewhere in the scientific literature. Such methods are useful in creating aerosol compositions of CFS compositions.

CFS compositions may also be inserted into a delivery device, e.g., a nebulizer or atomizer or vaporizer, in different forms. For example, the CFS compositions can be part of a solution contained in such a delivery device. As used herein, the term “solution” includes a pharmaceutically acceptable carrier or diluent. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and may optionally be preserved against the contaminating action of microorganisms such as bacteria and fungi through the use of, for example, parabens, chlorobutanol, phenol, ascorbic acid, thimerosal, and the like. Solutions of the invention can be prepared by incorporating the CFS compositions in a pharmaceutically acceptable carrier or diluent and, as required, other ingredients enumerated above.

The timing of administration of CFS compositions will depend upon the type and severity of the disease, disorder, or injury being treated. In one embodiment, the CFS compositions are administered as soon as possible after diagnosis or surgical intervention. In another embodiment, CFS compositions are administered more than one time following diagnosis or surgical intervention. In certain embodiments, where surgery is required, the CFS compositions are administered at surgery. In still other embodiments, the CFS compositions are administered at as well as after surgery. Such post-surgical administration may take the form of a single administration or multiple administrations.

Support matrices, scaffolds, membranes and the like into which the CFS compositions can be incorporated or embedded include matrices which are recipient-compatible and which degrade into products which are not harmful to the recipient. Detailed information on suitable support matrices, etc. can be found in U.S. Pat. Nos. 8,058,066 and 8,088,732, both of which are incorporated herein by reference.

A “therapeutically effective amount” of a therapeutic agent within the meaning of the present invention will be determined by a patient's attending physician or veterinarian. Such amounts are readily ascertained by one of ordinary skill in the art and will enable preventing or treating nasal polyps when administered in accordance with the present invention. Factors which influence what a therapeutically effective amount will be include, the specific activity of the therapeutic agent being used, the extent of the surgical wound, the absence or presence of infection, time elapsed since the surgery, and the age, physical condition, existence of other disease states, and nutritional status of the patient. Additionally, other medication the patient may be receiving will effect the determination of the therapeutically effective amount of the therapeutic agent to administer.

### EXAMPLES

The following examples are put forth so as to provide those of ordinary skill in the art with a complete disclosure and description of how to make and use the compositions and methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees centigrade, and pressure is at or near atmospheric.

The following examples provide evidence of the anti-inflammatory and wound healing effects of ACCS is several different inflammatory disease states (mucosal/infected; skin (intact and lesioned); and cutaneous wound/infected), thus providing strong evidence for the broad applicability of ACCS to treat inflammatory diseases such as nasal polyps and rhinosinusitis.

### Example 1

### Inflammatory Model—Use of ACCS to Prevent Onset of Periodontal Disease in an Animal Model

Objective: The aim of this study was to evaluate the preventive role of ACCS in Porphyromonas gingivalis (P. gingivalis)-induced experimental periodontitis in rabbits

Methods: Eight New-Zealand White rabbits were distributed into 3 groups: 1. Untreated (n=2), 2. Control (unconditioned ACCS culture media) (n=3), and 3. ACCS (n=3). At baseline, all rabbits received silk ligatures bilaterally tied around mandibular second premolars under general anesthesia. The assigned test materials, ACCS or control, in volumes of 10 μL were topically applied to the ligated sites with a blunt needled-Hamilton Syringe from the time of ligature; control animals received ligature, but no treatment. Topical P. gingivalis-containing slurry (1 mL) was subsequently applied to induce the periodontal inflammation. The application of test materials and P. gingivalis continued for 6 weeks on an every-other-day schedule. At 6 weeks, following euthanasia, the mandibles were surgically harvested. Morphometric, radiographic and histologic evaluations were performed.

Results: Macroscopic evaluations including soft tissue assessments, crestal bone and infrabony measurements showed significant periodontal breakdown induced by P. gingivalis in control and no treatment groups at 6 weeks compared to historical ligature-alone groups (p=0.05, p=0.03, respectively). ACCS application significantly inhibited soft tissue inflammation and prevented both crestal bone loss and infrabony defect formation compared to untreated and control groups (p=0.01, p=0.05, respectively). Histologic assessments and histomorphometric measurements supported the clinical findings; ACCS treated animals demonstrated significantly less inflammation in soft tissue and less bone loss compared to the untreated and control groups (p=0.05).

Conclusions: Topical ACCS application prevents periodontal inflammatory changes and bone loss induced by P. gingivalis as shown both at clinical and histopathological level. ACCS has potential as a therapeutic approach for the prevention of periodontal diseases

### Example 2

### Inflammatory Model—Use of ACCS to Stop Progression of or Reverse Periodontal Disease in an Animal Model

Objective: The aim of this study was to evaluate the therapeutic actions of ACCS in the treatment of periodontitis induced by P. gingivalis.

Methods: The study was conducted using a two-phase rabbit periodontitis protocol: 1—Disease induction (6 weeks) and 2—Treatment (6 weeks). Periodontal disease was induced in 16 New-Zealand White rabbits by every-other-day application of topical P. gingivalis to ligatured mandibular premolars. At the end of Phase 1, 4 randomly selected rabbits were sacrificed to serve as the baseline disease group. For Phase 2, the remaining 12 rabbits were distributed into 3 groups (n=4), 1—Untreated, 2—Control (unconditioned ACCS culture media) and 3—ACCS treatment. At the end of Phase 2, morphometric, radiographic and histologic evaluations were performed on harvested mandibles.

Results: The baseline disease group exhibited experimental periodontitis evidenced by tissue inflammation and bone loss. At the end of Phase 2, the untreated group showed significant disease progression characterized by increased soft and hard tissue destruction (p=0.05). The tissue inflammation and bone loss was significantly reduced by topical ACCS compared to baseline disease and untreated groups (p=0.05; p=0.002, respectively). The control treatment also arrested disease progression compared to untreated group (p=0.01), but there was no improvement in periodontal health compared to baseline disease (p=0.4). Histopathological assessments revealed similar findings; ACCS stopped the progression of inflammatory process (p=0.003) and reversed bone destruction induced by P. gingivalis (p=0.008). The ACCS-treated group had minimal osteoclastic activity limited to crestal area compared to untreated and control groups, which showed a profound osteoclastogenic activity at the bone crest as well as at interproximal sites.

Conclusions: Topical application of ACCS stopped the progression of periodontal inflammation and resulted in tissue regeneration in rabbit periodontitis indicating its potential therapeutic efficacy.

### Example 3

### Evaluate the Efficacy of Topically Applied ACCS to Inhibit Irritant 12-Otetradecanoylphorbol-3-acetate (TPA) Skin Inflammation in Mice

Method: Topical treatment was given twice daily to the following groups: 1. TPA+topical control; 2. TPA+ACCS; 3. TPA+clobetasol 0.05 topical solution (the strongest available topical corticosteroid); 4. ACCS alone; 5. No treatment (the other untreated ear was measured). The endpoints for the study were ear thickness and ear weight at the end of the experiment. The thicker the ear and the more it weighs correlates with the degree of inflammation.

Results: Topically applied ACCS was effective at reducing the inflammation induced by TPA. The anti-inflammatory activity of topical ACCS reached the same level as clobetasol (a class 1 potent topical corticosteroid) by 3 days after beginning application.

Conclusion: ACCS has a strong anti-inflammatory effect when applied to skin.

### Example 4

### Evaluate the Efficacy of Intralesional Injection of ACCS to Inhibit Irritant (TPA) Skin Inflammation in Mice

Method: Intralesional injection into the ear was given once daily to the following groups: 1. TPA+intralesional control; 2. TPA+intralesional ACCS; 3. TPA+intralesional kenalog (10 mg/ml) (a potent intralesional corticosteroid); 4. ACCS intralesional injection alone; 5. Saline sham injections to the normal untreated ear. The endpoints for the study were ear thickness and ear weight at the end of the experiment. The thicker the ear and the more it weighs correlates with the degree of inflammation.

Results: Intralesional injection of ACCS was effective at reducing the inflammation induced by TPA at all time points beginning on day 2 of daily injections. Intralesional kenalog (10 mg/ml) injections induced a hematoma at the site of injection, which led to some inflammation and that is why there is not a substantial difference in ear thickness when comparing TPA+kenalog with TPA+control.

Conclusions: Intralesional ACCS did reduce skin inflammation but the topically applied ACCS in Example 1 above had a more potent effect. There was no difference in ear weight using either ACCS or intralesional kenalog compared with TPA+control.

### Example 5

### Effects of ACCS in an Animal Model of Chronic Wound Healing

An art-accepted animal model for chronic granulating wound was used to study the effects of ACCS on chronic wound healing (Hayward P G, Robson M C: Animal models of wound contraction. In Barbul A, et al: Clinical and Experimental Approaches to Dermal and Epidermal Repair: Normal and Chronic Wounds. John Wiley & Sons, New York, 1990.).

Results: ACCS was effective in not allowing proliferation of tissue bacterial bioburden. ACCS allowed accelerated healing of the granulating wound significantly faster than the non-treated infected control groups.

### Example 6

### Effects of ACCS on In Vitro Protein Expression

The effects of ACCS is evaluated in in vitro models of nasal polyps by measuring the upregulation of mesenchymal-epithelial transition factor, periostin, and protein phosphatase 1 regulatory subunit 9B. TGFβ1 has also been implicated in the occurrence of non-allergic nasal polyps. (Platt et al, Current Opinion in Allergy and Clinical Immunology, 2009, 9:23-28).

### Example 7

### Effects of ACCS in an Animal Model of Nasal Polyps and Rhinosinusitis

The effects of ACCS is evaluated in animal models of nasal polyps. Suitable animal models are described in, for example, Bernstein, J. M., et al., Ann Otol Rhinol Laryngol, 2009, 118(12):866-875, Human nasal polyp microenvironments maintained in a vialble and functional state as xenografts in NOD-scid IL2rγnull mice; and Cho, S. H., et al., PLoS ONE, 2012, 7(4):e35114, Spontaneous eosinophilic nasal inflammation in a genetically-mutant mouse: comparative study with an allergic inflammation model.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification

1.-16. (canceled)

17. A method for treating or preventing nasal polyps in a patient in need thereof comprising administering to the patient a therapeutically effective amount Amnion-derived Cellular Cytokine Solution (ACCS).

18. The method of claim 17 wherein the ACCS is formulated for intranasal administration.

19. The method of claim 18 wherein the intranasal administration is aerosol or spray administration.

20. The method of claim 17 wherein the ACCS is contained in a surgical packing material.

21. A method for treating or preventing rhinosinusitis in a patient in need thereof comprising administering to the patient a therapeutically effective amount of ACCS.

22. The method of claim 21 wherein the ACCS is formulated for intranasal administration.

23. The method of claim 22 wherein the intranasal administration is aerosol or spray administration.

24. The method of claim 21 wherein the ACCS is contained in a surgical packing material.
